(19) United States
(12) Patent Application Publication
(10) Pub. No.: US 2006/0247655 A1
(43) Pub. Date: Nov. 2, 2006

(54) Instrument to insert a prosthetic implant

(75) Inventors: Tom J. Francis, Cordova, TN (US); Randy Allard, Germantown, TN (US); Hai H. Trieu, Cordova, TN (US); Chris Hughes, Lakeland, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/118,383

(22) Filed: May 2, 2005

(51) Int. Cl. A61F 2/46

(52) U.S. Cl. 606/99

(57) ABSTRACT

Devices and methods to insert a prosthetic implant into an intervertebral disc space. The device may comprise pivotally connected channel members and a locking mechanism pivotally connected to the channel members. The locking mechanism may comprise a latch that engages a slot in the sidewalls of a channel member to at least partially cover an open side of the channel members and lock the channel members in place. A prosthetic implant may be loaded into the channel members in a relaxed configuration and then unfolded to an implantation configuration by pivoting the channel members.

### FIELD OF THE INVENTION

Embodiments of the invention relate to devices and methods to insert a prosthetic implant into an intervertebral disc space, and more particularly to devices and methods for implanting a prosthetic implant to augment or repair an intervertebral disc space.

### DESCRIPTION OF THE RELATED ART

The intervertebral disc functions to stabilize the spine and to distribute forces between vertebral bodies. The intervertebral disc is composed primarily of three structures: the nucleus pulposus, the annulus fibrosis, and two vertebral end-plates. The nucleus pulposus is an amorphous hydrogel in the center of the intervertebral disc. The annulus fibrosis, which is composed of highly structured collagen fibers, maintains the nucleus pulposus within the center of the intervertebral disc. The vertebral end-plates, composed of hyalin cartilage, separate the disc from adjacent vertebral bodies and act as a transition zone between the hard vertebral bodies and the soft disc.

Intervertebral discs may be displaced or damaged due to trauma, disease, or the normal aging process. One way to treat a displaced or damaged intervertebral disc is by surgical removal of a portion or all of the intervertebral disc, including the nucleus and the annulus fibrosis. However, the removal of the damaged or unhealthy disc may allow the disc space to collapse, which can lead to instability of the spine, abnormal joint mechanics, nerve damage, and severe pain. Therefore, after removal of the disc, a prosthetic implant such as a prosthetic nucleus, artificial disc, or fusion cage may be implanted in order to replace the removed nucleus or annulus, or a portion thereof.

Prosthetic implants may be designed to be implanted in an insertion configuration, and then transform following insertion. For example, some prosthetic implants may be placed in an intervertebral disc space in an insertion configuration with a small cross section, and then transform, for example by unfolding, swelling, or otherwise expanding, to a configuration with a larger cross section. In this way, the implantation of the prosthetic implant is eased because of the decreased cross section of the disc during insertion into the intervertebral disc space.

The description herein of problems and disadvantages of known devices, methods, and apparatuses is not intended to limit the invention to the exclusion of these known entities. Indeed, embodiments of the invention may include one or more of the known devices, methods, and apparatuses without suffering from the disadvantages and problems noted herein.

### BRIEF SUMMARY OF THE INVENTION

What is needed are devices and methods for inserting a prosthetic implant into an intervertebral disc space. Additionally, there is a need for an insertion device that may transform a prosthetic implant to an insertion configuration with a reduced cross section in order to ease its implantation and to reduce injury to the disc during insertion (i.e., forming a smaller hole for insertion). Additionally, there is a need for an insertion device that is simple and convenient to use. Embodiments of the invention solve some or all of these needs, as well as additional needs.

Therefore, in accordance with an embodiment of the invention, there is provided an instrument for inserting a prosthetic implant, comprising first and second channel members. The first channel member comprises first and second ends, a bottom, two sidewalls, and an open side. The second channel member comprises first and second ends, a bottom, two sidewalls, an open side, and a slot in the top of the sidewalls positioned along its longitudinal extent. The first and second channel members may be pivotally connected at their first ends and define a lumen extending therethrough. The device also may comprise a locking mechanism capable of at least partially covering the open sides of the first and second channel members. The locking mechanism may be pivotally connected to the first channel member and have a latch capable of engaging the slot in the sidewalls of the second channel member.

In accordance with another embodiment of the invention, there is provided a method of inserting a prosthetic implant into an intervertebral disc space. A device as described in embodiments of the invention may be provided. The first and second channel members of the device may be pivoted to a position substantially less than 180 degrees and a prosthetic implant may be loaded into the channel members. The first and second channel members then may be pivoted to a position of approximately 180 degrees and the latch of the locking mechanism may be engaged with the slot in the second channel member in order to lock the channel members in place. The prosthetic implant then may be advanced through the first and second channel members and into the intervertebral disc space.

In accordance with another embodiment of the invention, there is provided a device comprising first and second channel members and a locking mechanism as described herein. Additionally, the device may comprise a tip and a ratchet mechanism detachably connectible to the first and second channel members, respectively.

In accordance with another embodiment of the invention, there is provided a method of inserting a prosthetic implant into an intervertebral disc space using a device comprising first and second channel members, a locking mechanism, a tip, and a ratchet mechanism. A prosthetic implant may be loaded into the channel members as described. Then, the tip and ratchet mechanism may be connected to the device. The distal end of the tip may be placed in an intervertebral disc space and the ratchet mechanism may be activated in order to push the prosthetic implant into the disc space.

These and other features and advantages of the present invention will be apparent from the description provide herein.

### BRIEF DESCRIPTION OF THE DRAWINGS

FIG. 1 is an illustration of an exemplary device according to embodiments of the invention.

FIG. 2, embodiments A, B, and C, is an illustration of an exemplary prosthetic implant that may be inserted into an intervertebral disc space using devices and methods of the invention.

FIG. 3 is an illustration of first and second channel members according to embodiments of the invention.

FIG. 4 is an illustration of first and second channel members and a locking mechanism in a latched position according to embodiments of the invention

FIG. 5 is an illustration of a ratchet mechanism according to embodiments of the invention.

FIG. 6 is an illustration of a tip of the device according to embodiments of the invention.

FIG. 7 is an illustration of a device in a loading position according to embodiments of the invention.

FIG. 8 is an illustration of a prosthetic implant loaded into a device according to embodiments of the invention.

FIG. 9, embodiments A, B, and C, is an illustration of a prosthetic implant being delivered to an intervertebral disc space according to methods of the invention.

### DETAILED DESCRIPTION OF THE INVENTION

The following description is intended to convey a thorough understanding of the various embodiments of the invention by providing a number of specific embodiments and details involving devices and methods for inserting a prosthetic implant into an intervertebral disc space. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the expression “intervertebral disc space” refers to any volume or void between two adjacent vertebrae. The intervertebral disc space may be the volume inside of the annulus fibrosis of the intervertebral disc. Alternatively, the intervertebral disc space also may include the annulus fibrosis itself. The intervertebral disc space also may include a portion or the entire volume inside the annulus fibrosis.

The expression “prosthetic implant” refers to any applicable implantable device including, but not limited to, nucleus pulposus replacements, replacement intervertebral discs, fusion devices such as fusion cages, and the like. The prosthetic implant may comprise any number of synthetic, as well as natural or endogenous, materials.

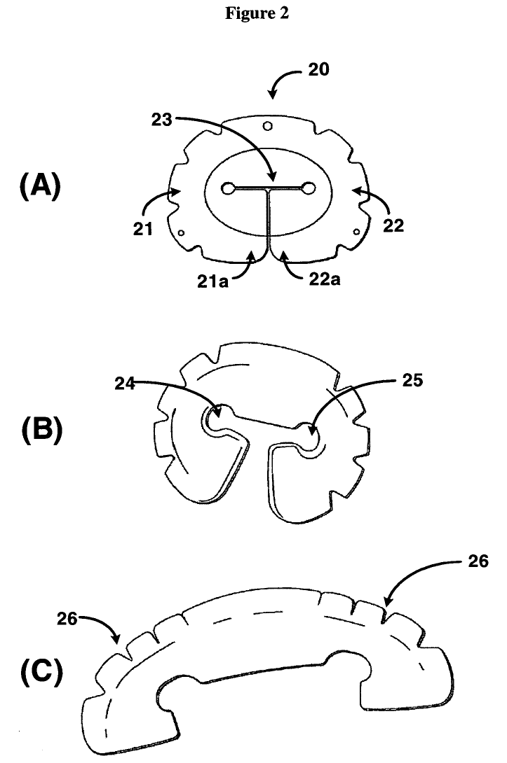

It is a feature of an embodiment of the present invention to provide an instrument to insert a prosthetic implant. Devices according to embodiments of the invention may be useful to insert a prosthetic implant such as the exemplary prosthetic implant shown in FIG. 2, embodiments A, B, and C. In embodiment A, the prosthetic implant 20 is in a relaxed or folded configuration such as that preferably obtained following implantation of the prosthetic implant. The relaxed or folded configuration corresponds to a configuration where the prosthetic implant 20 can function, for example, as an artificial intervertebral disc, a nucleus replacement, or a spinal fusion device. The prosthetic implant 20 comprises a pair of arms 21 and 22 that are folded to form an inner fold 23 and abut one another at their ends 21a and 22a when the disc is in a relaxed configuration.

In embodiment B, the prosthetic implant 20 is shown in a less relaxed, slightly straightened configuration where the two arms have been bent away from each other. The two arms may be bent away from each other by the application of an external force. For example, posts on an inserter instrument for inserting the prosthetic implant may engage apertures 24 and 25 of the disc in order to transfer a force from the instrument to unfold the prosthetic implant's arms. As the instrument is operated or activated, the posts thereon are separated from each other, thereby causing the prosthetic implant 20 to open to a more straightened configuration. Grooves 26 on the outer surface of the prosthetic implant may be provided to prevent cracking or tearing of the disc as it is deformed to an even more straightened configuration, as shown in embodiment C.

In general, a straightened configuration of the prosthetic implant corresponds to an implantation configuration because the prosthesis has a smaller cross sectional area when it is in a straightened configuration. As can be seen, the cross sectional size of the straightened prosthetic implant shown in embodiment C is less that the cross sectional size of the relaxed, folded prosthetic implant shown in embodiment A. Therefore, the prosthetic implant in a straightened configuration may be easier to implant into an intervertebral disc space, especially if implantation is to be carried out by minimally invasive surgical techniques through a small incision or defect in the annulus. As the prosthetic implant 20 is opened to a more straightened configuration, the disc becomes less relaxed (i.e. more strained). Upon release of the external force, for example the external force applied by an inserter instrument, the straightened prosthetic implant preferably may resume its folded configuration, wherein the prosthetic implant can act, for example, as an artificial intervertebral disc, a nucleus replacement, or a spinal fusion device.

An instrument for inserting a prosthetic implant according to embodiments of the invention may comprise a first channel member having first and second ends, a bottom, two sidewalls, and an open side. Additionally, the instrument may comprise a second channel member having first and second ends, a bottom, two sidewalls, an open side, and a slot in the top of the sidewalls positioned along its longitudinal extent. The first and second channel members may form a three sided lumen or passageway extending through both the channel members. This lumen or passageway may be useful for placement and passage of a prosthetic implant, including, but not limited to, a prosthetic implant of the type described herein. Accordingly, the lumen or passageway preferably may be smooth on its interior surface. Ridges, indentations, projections, etc., may be provided on the interior surface to the extent they assist in, or at least do not prevent, passage of the prosthetic implant through the lumen or passageway formed of the first and second channel members.

Preferably, the first and second channel members may form a lumen or passageway having an inner diameter of between about 2 millimeters to about 20 millimeters, with an inner diameter of between about 5 millimeters and about 10 millimeters being more preferred. The length of the lumen or passageway formed by the first and second channel members preferably may be between about 5 centimeters and about 30 centimeters, with a length of between about 10 centimeters and about 25 centimeters being more preferred.

The first and second channel members may be pivotally connected at their first ends using a pin, hinge, or some other appropriate connection. Preferably, the first and second channel members may be pivotally connected in a fashion wherein they can be unfolded to an angle of approximately 180 degrees, or a straight position. It also is preferred that the first and second channel members are pivotally connected in a fashion wherein they can be folded to an angle of substantially less than 180 degrees, for example to 90 degrees or less.

The approximately 180 degree, or straight position, may correspond to an insertion configuration wherein a prosthetic implant that has been placed in the first and second channel members is unfolded to a configuration having a substantially reduced cross section. As mentioned, a decreased cross section may aid in delivery of the prosthetic implant to the intervertebral disc space. The position where the first and second channel members are at an angle with respect to each other of substantially less than 180 degrees may correspond to a position for loading of the prosthetic implant in the instrument. This may be advantageous for a prosthetic implant that assumes a folded configuration when it is in a relaxed state. In this manner, the prosthetic implant may be loaded into the instrument while in a relaxed state, thereby easing loading of the prosthetic implant. Then, the first and second channel members may be unfolded to an approximately 180 degree, or straight position, in order to unfold the prosthetic implant to an insertion configuration and thereafter insert the prosthesis into an intervertebral disc space.

In a preferred embodiment, the first channel member of the instrument may comprise a post extending inward from a sidewall of the first channel member. Additionally, the second channel member also may comprise a post extending inward from a sidewall of the second channel member. The sidewalls of the first and second channel members having posts extending inward therefrom may be on the same side of the instrument. However, it should be recognized that the configuration of the posts may differ depending upon the nature of the prosthetic implant for which the insertion instrument is designed.

These posts may be useful to engage a prosthetic implant that is to be placed in the instrument. For example, the posts may engage apertures in a prosthetic implant such as the exemplary device illustrated in FIG. 2. Accordingly, the posts may be located anywhere along the longitudinal extent of the sidewalls of the first and second channel members so as to engage apertures in a prosthetic implant. For example, in order to engage apertures in a prosthetic implant such as is illustrated in FIG. 2, it may be preferable to locate the posts near the first ends of the first and second channel members. The posts also may be useful to cause the prosthetic implant to unfold when the first and second channel members are brought to an approximately 180 degree, or straight, position. The prosthetic implant may unfold because the distance between the two posts may increase as the first and second channel members are opened from an angle substantially less than 180 degrees to an angle of approximately 180 degrees.

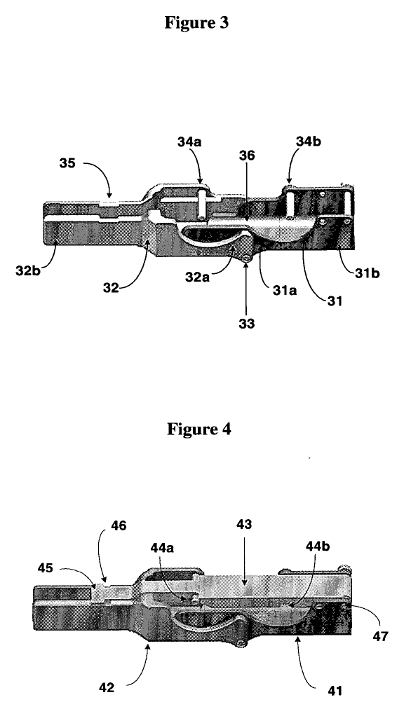

FIG. 3 is a drawing of exemplary first and second channel members according to embodiments of the invention. A first channel member 31 has a first end 31a and second end 31b. A second channel member 32 also has a first end 32a and second end 32b. The first and second channel members are pivotally connected at their first ends using, for example, a pin 33. Posts 34a and 34b extend inward from adjacent sidewalls of the first and second channel members. The first and second channel members form a lumen or passageway 36, where a prosthetic implant may be placed.

The second channel member 32 preferably has a slot, notch, or detent positioned at the top of its sidewalls somewhere along its longitudinal length. For purposes of illustration only, a slot 35 is positioned at the top of the sidewalls of the second channel member shown in FIG. 3. The slot may be useful to engage a locking mechanism that also preferably may be part of the instrument.

The locking mechanism may be described as a plate at least partially covering the open sides of the first and second channel members and may be pivotally connected to the first channel member, having a latch that engages the slot, notch, or detent in the sidewalls of the second channel member. The locking mechanism may be of a length such that it can engage the slot, notch, or detent in the sidewalls of the second channel member only when the first and second channel members are in an approximately 180 degree, or straight position. The locking mechanism is in a closed position when the first and second channel members are in an approximately straight position and the locking member is latched. When the first and second channel members are at an angle of substantially less than 180 degrees, or a loading position, the locking mechanism preferably will not be able to close.

The locking mechanism may be useful to lock the instrument so that the instrument maintains the angle of approximately 180 degrees, thereby keeping the implant in a straightened configuration and thus facilitating implantation. If optional posts are included on sidewalls of the first and second channel members, the locking mechanism may have indents so that when the locking mechanism is in a closed position the posts on the sidewalls of the first and second channel members may rest in the indents.

FIG. 4 is a drawing of first 41 and second 42 channel members and a locking mechanism 43 according to embodiments of the invention. The locking mechanism 43 is pivotally connected to the first channel member 41 using, for example, a pin 47. The latch 45 of the locking member 43 engages a slot 46 in the second channel member 42. Optional indents 44a and 44b in the locking mechanism serve as receptacles for optional posts in the sidewalls of the channel members. The first and second channel members are shown in an approximately 180 degree, or straight, position. It will be appreciated that the latched locking mechanism secures or holds the first and second channel members in an approximately straight position. To fold the instrument into a loading position where the angle between the first and second channel members is substantially less than 180 degrees, the locking mechanism may be unlatched and pivotally displaced, after which the first and second channel members may be pivotally displaced to an angle substantially less than 180 degrees to attain a loading position.

A ratchet mechanism may be attached to the second end of the second channel member in such a way as to prevent the latched locking mechanism from being unlatched. The ratchet mechanism of the instrument may work to push the prosthetic implant through the first and second channel members and into an intervertebral disc space. An exemplary ratchet mechanism may operate in a manner similar to a caulking gun.

For example, in a preferred embodiment, the ratchet mechanism may comprise a handle, a lever pivotally attached to the handle, two sets of clutch plates mounted on the handle, and a plunger engageable by the clutch plates. By “sets of clutch plates,” it is meant that each set of clutch plates contains at least one clutch plate, but also may contain two or more clutch plates. The clutch plates may be apertured so that the plunger can pass through the apertures in both sets of clutch plates. The clutch plates, when they are in a position approximately perpendicular to the plunger, preferably do not engage the plunger. In contrast, when the clutch plates are canted relative to the plunger, they preferably frictionally engage the plunger. When the ratchet mechanism is resting, each set of clutch plates may assume a certain orientation (i.e. canted or approximately perpendicular) relative to the plunger. When the ratchet mechanism is activated, each set of clutch plates may assume a different orientation relative to the plunger.

One set of clutch plates may be driving clutches. The driving clutches may be approximately perpendicular to the plunger in a resting position. In other words, the driving clutches preferably do not engage the plunger when they are in a resting position. When the ratchet mechanism is activated, for example by operating the lever, the driving clutches may become canted so that they frictionally engage the plunger. Activating the ratchet mechanism also may cause the driving clutches to move forward, and because the driving clutches are in frictional engagement with the plunger, the plunger also may be advanced in a forward, or distal, direction.

The other set of clutch plates may be holding clutches. The holding clutches may be canted relative to the plunger when the ratchet mechanism is in a resting position. In other words, the holding clutches in a resting position preferably frictionally engage the plunger. This may be advantageous so as to prevent the plunger from backing out of the ratchet mechanism after being advanced by the driving clutches. Preferably, the holding clutches extend beyond the body of the handle so that the operator can manually release the holding clutches in order to remove the plunger when desired.

Preferably, the holding clutches are mounted in such a way that advancing the plunger by activating the ratchet mechanism automatically moves the holding clutches to an activated position where the holding clutches are approximately perpendicular to the plunger. Therefore, the holding clutches preferably do not frictionally engage the plunger in the activated position, or at least will not prevent the plunger from advancing in a forward or distal direction when the ratchet mechanism is activated. In this manner, the holding clutches will not prevent the plunger from being advanced by the driving clutches when the ratchet mechanism is activated, for example by operating the lever. Upon release of the driving clutches, the holding clutches preferably may resume their resting, canted position so as to frictionally engage the plunger, thereby preventing the plunger from backing out of the ratchet mechanism.

The plunger may be a longitudinal rod having proximal and distal ends and a circular cross section. In a preferred embodiment, the plunger may be longitudinally notched such that its cross section, along at least a portion of its longitudinal axis, is not perfectly circular. A cross section in this style may help to ensure that the clutch plates are better able to frictionally engage the plunger. The proximal end of the plunger may be bent, for example at about a 90 degree angle, in order to provide a grip to facilitate grasping, inserting, and removal of the plunger into the ratchet mechanism. At the distal end of the plunger, it may have a blunt nosed cover. The blunt nosed cover may help the plunger to engage the prosthetic implant in the first and second channel members so that the plunger's distal movement when the ratchet mechanism is activated is translated into distal movement of the prosthetic implant.

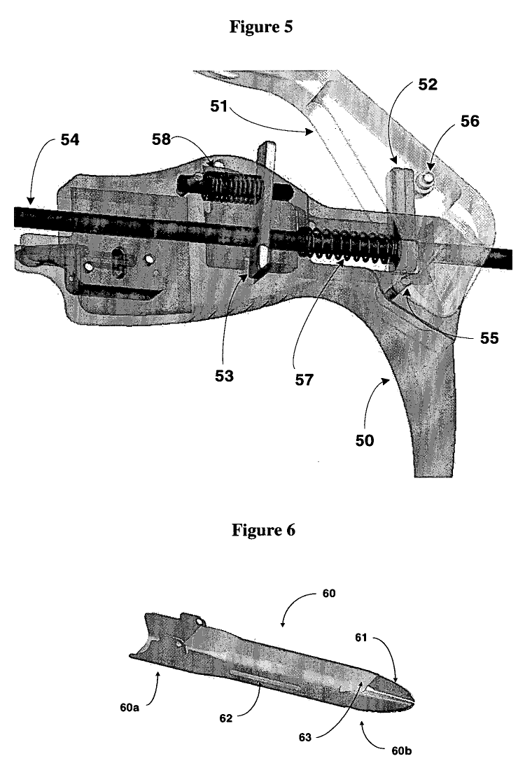

An exemplary ratchet mechanism according to embodiments of the invention is illustrated in FIG. 5. A handle 50, a lever 51, a first set of clutch plates that are driving clutches 52, a second set of clutch plates that are holding clutches 53, and a plunger 54 are provided. The lever 51 is attached to the handle 50 by a pivot or pin 55. The driving clutches 52 and holding clutches 53 are attached to the handle 50. The plunger passes through apertures or holes in the driving and holding clutches. The lever comprises a hollowed-out portion with a cam 56 disposed therein. The ratchet mechanism may be activated by pivoting the lever 51 towards the handle 50 so that the cam 56 engages the driving clutches 52.

In a resting position, a spring 57 biases the driving clutches 52 to a position approximately perpendicular to the plunger 54 such that the driving clutches do not frictionally engage the plunger. As is exemplarily illustrated, the spring 57 biasing the driving clutches 52 may be positioned coaxial to the plunger 54 with one end connected to the handle 50 and the other end connected to the driving clutches 52. The ratchet mechanism may be activated by pivoting the lever 51 so that cam 56 engages the driving clutches 52, whereby the driving clutches 52 are canted so that they frictionally engage the plunger 54. Continued pivoting of the lever 51 may push the driving clutches 52 forward so that the plunger 54 is advanced in a forward, or distal, direction.

The holding clutches 53 may be positioned elsewhere in the handle 50. A spring 58 attached at one end to the handle 50 and at the other end to the holding clutches 53 may bias the holding clutches to be canted relative to the plunger 54 in a resting position. In other words, the spring 58 may cause the holding clutches 53 to frictionally engage the plunger 54 when the ratchet mechanism is in a resting position.

When the ratchet mechanism is activated so that the plunger 54 is forwardly or distally advanced, the holding clutches 53 preferably may be pivoted by the forward action of the plunger to an approximately perpendicular position relative to the plunger so that the holding clutches no longer frictionally engage the plunger. When the ratchet mechanism is no longer activated, forward or distal movement of the plunger 54 stops and the holding clutches 53 may be returned to a canted position by action of the spring 58. In this way, the holding clutches 53 serve to prevent the plunger from backing out of the ratchet, or moving proximally, but do not prevent the plunger from advancing forward, or distally.

The holding clutches 53 preferably extend beyond the handle 50 to which they are mounted. This may allow an operator to manually disengage the holding clutches 53 in order to release and withdraw the plunger 54. In other words, an operator can manually move the holding clutches 53 to a position approximately perpendicular to the plunger 54 so that the clutches do not frictionally engage the plunger; the plunger then may be removed from the ratchet mechanism.

A tip may be connected to the second end of the first channel member for dilating an incision or defect in an annulus. In other words, the tip may be designed to be effective for dilating a small opening in a disc annulus so that the opening is made large enough for the prosthetic implant being implanted to pass through the tip and into the intervertebral disc space. Dilating a hole or fissure in an annulus may be especially useful if the prosthetic implant to be implanted is a prosthetic nucleus replacement and at least a portion of the natural annulus fibrosis is to be retained following implantation of the prosthetic nucleus. Preferably, the dilator is capable of dilating the opening without tearing the annulus, so that the dilated opening shrinks back to a smaller size after the tip of the instrument is removed from the intervertebral disc space.

The tip may comprise distal and proximate ends and four side walls defining a lumen. The proximate end of the tip may be connected to the second end of the first channel member so that the tip's lumen is in communication with the lumen formed by the first and second channel members. One or more small flexible arms may be located at the distal end of the tip. The arms may be designed to facilitate dilating a small incision or defect in the annulus so that the incision or defect can temporarily be made large enough to allow passage of the prosthetic implant. In a resting state, the arms preferably may point inwards towards the lumen of the tip. When a prosthetic implant is passed through the tip, however, the arms preferably may flex or bend outwards, away from the lumen in the tip. In this manner, passage of a prosthetic implant through the tip may cause the arms at the distal end of the tip to spread and dilate an incision or defect in the annulus. Such a tip can be described as a “passive” dilator, because it is operated indirectly via the passing prosthetic implant, not by direct actuation.

In a preferred embodiment, the tip additionally may comprise a guiding arm also positioned at the distal end of the tip. The guiding arm may be generally shorter than the other arms. Also, the guiding arm may be generally stiffer than the other arms. Because the guiding arm is shorter than the other arms, a prosthetic implant may be guided to fold towards the guiding arm upon exiting the tip. Therefore, in order to guide the direction in which a prosthetic implant may fold upon insertion into the intervertebral disc space, the guiding arm may be oriented in the direction in which folding is desired.

The tip additionally may comprise a window, aperture, or opening in at least one of the four side walls that define the lumen in communication with the lumen formed by the first and second channel members. The window may be designed to allow observation of the prosthetic implant as it is passed through the tip. In this manner, an operator of the instrument may observe the passage of the prosthetic implant through the tip and into the intervertebral disc space.

FIG. 6 is a drawing of an exemplary tip 60 according to embodiments of the invention. The proximate end of the tip 60a can be connected to the second end of the first channel member. At the distal end 60b of the tip, one or more arms 61 are found, and preferably at least four arms are utilized. In a resting position, as can be seen in the figure, the arms are biased towards the lumen in the tip, or at least extend straight out from the walls of the tip. When the arms are activated by a prosthetic implant passing through them, the arms may bend or flex outwards from the lumen in the tip. In this way, the arms may work to spread or dilate an incision or defect in the annulus, thereby easing passage of the prosthetic implant into the intervertebral disc space.

A guiding arm 63 also may be provided at the distal end 60b of the tip. The guiding arm 63 may be generally stiffer and shorter than the other arms 61 so as to aid in guiding the prosthetic implant as it exits the distal end of the tip. In a preferred embodiment, because the guiding arm 63 may be shorter than the other arms 61, the prosthetic implant folds in the direction of the guiding arm 63 as it exits the distal end of the tip. In this way, the folding of a prosthetic implant can be directed by orienting the guiding arm 63 in the direction in which folding is desired. An aperture or window 62 also is provided in a sidewall of the tip by which the operator can observe the passage of the prosthetic implant.

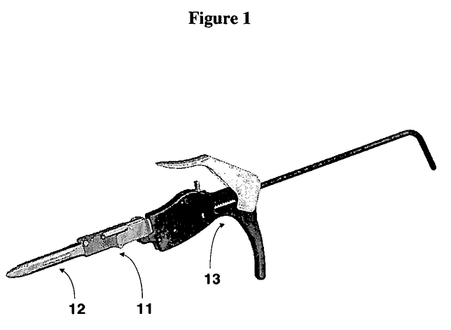

FIG. 1 is a drawing of an assembled instrument according to embodiments of the invention. The first and second channel members 11 form the center portion of the instrument. The ratchet mechanism 13 is attached to the second channel member, and the tip 12 is attached to the first channel member. To use the device, at least the distal end of the tip of the instrument may be inserted into an intervertebral disc space with the instrument in the depicted position where the first and second channel members art at an angle of approximately 180 degrees. A previously loaded prosthetic implant may be forced though the first and second channel members, the tip, and into the disc space by action of the ratchet mechanism. Preferably, the prosthetic implant, upon exiting the inserter instrument and entering the intervertebral disc space, transforms from a straightened configuration to a folded or resting configuration.

The instrument described herein may be made from a variety of materials, including, for example, medical plastics such polyvinyl chlorides, polypropylenes, polystyrenes, acetal copolymers, polyphenyl sulfones, polycarbonates, acrylics, silicone polymers, and mixtures and combinations thereof. Medical alloys such as titanium, titanium alloys, tantalum, tantalum alloys, stainless steel alloys, cobalt-based alloys, cobalt-chromium alloys, cobalt-chromium-molybdenum alloys, niobium alloys, and zirconium alloys also may be used to fabricate the instrument.

In another embodiment of the present invention, a method for implanting a prosthetic implant is provided. Instruments may be provided as described herein, for example that comprise first and second channel members pivotally connected at their first ends and a locking mechanism pivotally connected to the first channel member, where a latch on the locking mechanism can engage a slot at the top of the second channel member's sidewalls. The instrument additionally may comprise a tip and a ratchet mechanism, as described herein.

To load a prosthetic implant into the first and second channel members, the ratchet mechanism, and optionally the tip also, preferably may be detached from the first and second channel members. This may be advantageous because the tip and ratchet mechanism may add unnecessary bulk during the loading process. Additionally, if the ratchet mechanism and locking mechanism are configured such that the locking mechanism cannot open when the ratchet mechanism is attached to the second channel member, the ratchet mechanism may need to be detached so that the locking mechanism may be freely unlatched and latched during loading of the prosthetic implant.

The first and second channel members may be pivoted so that they are oriented at an angle substantially less than 180 degrees with respect to one another. This may be referred to as a loading position, because the angled relationship may facilitate loading of the prosthetic implant into the instrument. In a preferred embodiment, apertures, slits, detents, or another such portion or feature of the prosthetic implant may engage posts on the sidewalls of the first and second channel members. The posts therefore may serve to secure the prosthetic implant in the first and second channel members.

Upon loading of the prosthetic implant into the first and second channel members, the channel members may be pivoted or unfolded to an approximately 180 degree, or straight, position. This position may correspond to an implantation position because, by pivoting the first and second channel members to an approximately straight position, the prosthetic implant loaded therein also may be straightened to a configuration that facilitates insertion of the prosthetic implant into an intervertebral disc space. For example, the prosthetic implant in a straightened configuration may have a smaller cross section so that it can fit through a small tear, hole, or other defect in the annulus.

The locking mechanism then may be closed in order to lock or secure the channel members in the approximately straight position. The locking mechanism may be closed, for example, by engaging a latch thereon with slots in the sidewalls of the second channel member. This may be desirable because the prosthetic implant placed inside of the channel members, which has been deformed to a straightened configuration, may be under tension such that it would cause the channel members to pivot or fold back to a loading position if it where not for the channel members being restrained by the locking mechanism.

Then, the ratchet mechanism may be attached to the second channel member. In a preferred embodiment, the ratchet mechanism and locking mechanism may be configured in such a way that attaching the ratchet mechanism to the second channel member will prevent the latch of the locking mechanism from disengaging from the slots in the second channel member.

Also, the tip may be connected to the first channel member, if it has not yet been attached, for example, during loading of the prosthetic implant into the channel members.

To implant the prosthetic implant, a small incision (i.e., “aperture,” “opening,” or “portal”) may be cut in the annulus of the intervertebral disc space being repaired or augmented. In another alternative, the prosthetic implant may be inserted through an already existing defect in the annulus; therefore, no incision would be needed. A guide wire or other small instrument may be used to make the initial incision and to locate the point of entry into the disc space, if desired. If necessary, successively larger incisions may be cut from an initially small puncture. The incision or defect may allow passage of the prosthetic implant, so that the prosthetic implant can be implanted into the intervertebral disc space through the annulus. Preferably, the incision or defect is as small as possible in order to minimize the chance of expulsion of the prosthetic implant through the incision after the surgery is complete.

Once an incision is provided, or alternatively through an existing defect in the annulus, the distal end of the tip of the instrument may be inserted into or placed at the incision or defect in the annulus. Because the tip of the instrument has arms that are angled inward towards the tip's lumen, the tip may be used to partially dilate the incision or defect in the annulus. The tip preferably stretches the incision or defect temporarily, and avoids tearing so that the incision or defect can return back to its undilated size after the instrument is removed. Even if some tearing or permanent stretching occurs, the dilation preferably is accomplished in a manner that allows the incision or defect to return to a size smaller than its dilated size after the surgery is complete.

If it has not been done so already, the plunger may be inserted into the ratchet mechanism. If the plunger has a non-circular cross section along a portion of its longitudinal length, care may be taken to ensure that the plunger is correctly oriented with respect to the two sets of clutch plates so that the clutch plates may correctly engage the plunger. The ratchet mechanism then may be activated, for example, by operating or moving the lever attached to the handle. In this manner, the plunger may be forwardly or distally advanced through the ratchet mechanism, thereby pushing the prosthetic implant in the first and second channel members into the tip and thenceforth into the intervertebral disc space.

If desired, before placing the tip of the instrument at or in the incision or defect in the annulus, the ratchet handle may be activated to advance the prosthetic implant to a position just before the arms at the distal end of the instrument's tip. This may minimize the time and travel required for insertion of the prosthetic implant once the instrument is installed at the operative site. If a prosthetic implant is accidentally advanced to the point where one or more arms at the end of the tip begin to open or dilate, the prosthetic implant may be extruded out of the device and the instrument reloaded.

As the prosthetic implant passes by the arms at the distal end of the tip, it may cause the arms to flex or bend outwards, thereby dilating the annulus in order to temporarily increase the size of the incision or defect in the annulus through which the prosthetic implant is passing into the intervertebral disc space. If the tip is provided with a guiding arm, the guiding arm may function to aid in directing folding of the prosthetic implant as it enters the intervertebral disc space. Preferably, the guiding arm may be shorter that the other arms so that the prosthetic implant folds towards the guiding arm upon exiting from the tip. The tip of the instrument also may be moved from side-to-side, or from front-to-back, as necessary to deliver the prosthetic implant to the intervertebral disc space.

After the prosthetic implant is delivered to the intervertebral disc space, the instrument may be withdrawn and the incision or defect in the annulus may be allowed to return to its original size. If the annulus has been stretched or torn so that it does not return to its original size, the incision or defect preferably may at least return to a size smaller than its dilated size.

During or upon delivery to the intervertebral disc space, the prosthetic implant preferably folds from a straightened configuration to a folded or relaxed configuration. The relaxed configuration may correspond to the long-term configuration of the prosthetic implant in the intervertebral disc space.

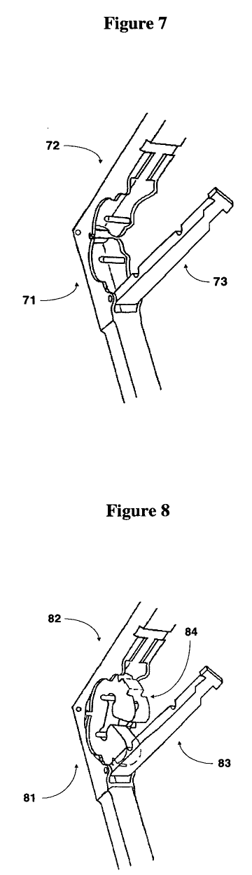

FIGS. 7-9 illustrate an exemplary method of implanting a prosthetic implant according to embodiments of the invention. FIG. 7 is a drawing of an exemplary loading position of a first and second channel member. As depicted, first 71 and second 72 channel members are at an angle relative to each other of substantially less than 180 degrees. This position may correspond to a loading position because it may enable a prosthetic implant to be loaded into the instrument while the prosthetic implant is in a relaxed, or folded, state. This may be desirable to make it easier to insert the prosthetic implant into the instrument. Additionally, the locking mechanism 73 is shown in an open state that allows loading of the prosthetic implant into the lumen of the first 71 and second 72 channel members.

FIG. 8 is a drawing of a prosthetic implant 84 loaded into the first 81 and second 82 channel members in a loading position with the locking mechanism 83 in an open state. Upon loading of the prosthetic implant into the first and second channel members, the members may be pivoted so as to attain an angle therebetween of approximately 180 degrees so that the first and second channel members are approximately straight. This position may correspond to an implantation position because it is in this position that the prosthetic implant will be implanted into an intervertebral disc space. It will be appreciated that when the first and second channel members are brought to an approximately straight position, the prosthetic implant that has been loaded inside of the channel members will be deformed, bent, or unfolded to an approximately straight configuration, as shown in, for example, FIG. 2, embodiment C. This configuration may result in the prosthetic implant attaining a shape with a smaller cross section so that the disc will be easier to implant into an intervertebral disc space, for example, through a small incision or defect in the annulus or using minimally invasive surgical techniques.

Upon pivoting the first and second channel members to an approximately straight position, the tip and ratchet mechanism of the instrument may be attached to the second end of, respectively, the first and second channel members. If desired, the ratchet mechanism may be activated in order to advance the prosthetic implant to a position just before the arms at the distal end of the instrument's tip. Those skilled in the art will appreciate that other devices may be substituted for the tip and ratchet mechanism described herein. For example, any tip or no tip may be attached, and any rod-like plunger could be used to advance the prosthetic implant into the disc space.

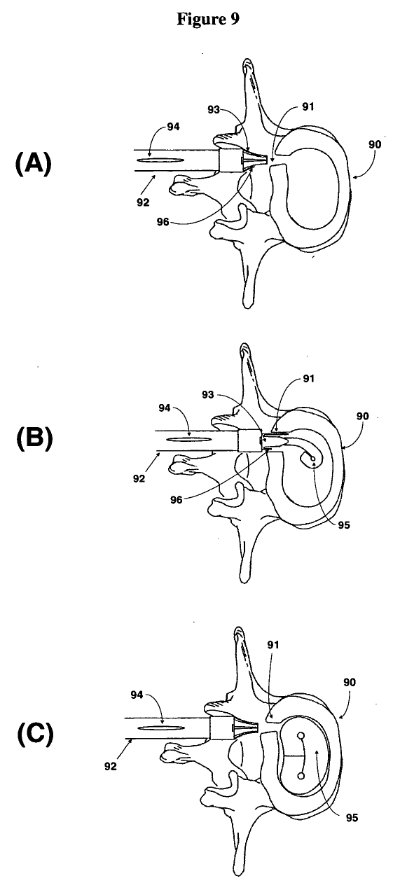

In FIG. 9, embodiment A, the distal end of the tip of an instrument according to embodiments of the invention is illustrated approaching an intervertebral disc space. The intervertebral disc space 90 has an incision or defect 91 in the annulus that will provide access so that a prosthetic implant may be implanted therein. The tip 92 of the instrument has arms 93 at its distal end that will be inserted into the incision or defect 91 in order to dilate the incision or defect when the prosthetic implant is passed through the tip of the instrument and into the intervertebral disc space. A guiding arm 96 also is positioned at the distal end of the tip adjacent the other arms 93. As depicted in FIG. 9, embodiment A, the arms 93 are in a resting position where they are generally bent or angled inward towards the lumen in the tip. The dilating arms will temporarily expand the incision or defect in the annulus so that the prosthetic implant can pass through. An optional aperture 94 also is provided in the tip of the instrument so that a surgeon or other operator can observe the passage of a prosthetic implant therethrough, if desired. In this way, the operator may know when the prosthetic implant has been delivered to the intervertebral disc space.

In FIG. 9, embodiment B, the prosthetic implant is depicted entering the intervertebral disc space 90. The arms 93 at the distal end of the tip 92 are dilated as the prosthetic implant 95 passes therethrough. As can be seen, while being inserted, the portion of the prosthetic implant that has left the confines of the instrument seeks to return to its relaxed state, in this case folding upon itself. A guiding arm 96 that also is positioned at the distal end of the tip adjacent the other arms 93 guides the prosthetic implant to fold in its direction because it is shorter than the arms 93 adjacent to it. Though not shown, it is to be understood that the prosthetic implant may be advanced into the tip of the instrument from the first and second channel members by activation of the ratchet mechanism, or other advancing mechanism, which may cause the plunger to impinge upon and push the prosthetic implant through the first and second channel members, the tip, and into the intervertebral disc space.

In FIG. 9, embodiment C, the prosthetic implant is depicted in a final, resting configuration. The prosthetic implant 95 has achieved its final configuration by folding to resume its resting, relaxed configuration upon insertion into the intervertebral disc space 90. Having delivered the prosthetic implant, the tip of the instrument 92 may be withdrawn from the intervertebral disc space.

Although FIG. 9 illustrates a posterior approach to an intervertebral disc space, it should be understood that any applicable surgical approach to the intervertebral disc space may be used, in accordance with the limitations described herein. Therefore, posterior, lateral, and transforaminal approaches also are contemplated by the present invention.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A device for inserting a prosthetic implant, comprising:
a first channel member having first and second ends, a bottom, two sidewalls, and an open side;
a second channel member having first and second ends, a bottom, two sidewalls, an open side, and a slot in the top of the sidewalls positioned along its longitudinal extent; and
a locking mechanism capable of at least partially covering the open sides of the first and second channel members and pivotally connected to the first channel member, having a latch capable of engaging the slot in the sidewalls of the second channel member;
wherein the first and second channel members are pivotally connected at their first ends and define a lumen extending through the first and second channel members.

2. The device of claim 1, wherein the first and second channel members are capable of being pivoted to a position wherein the angle between the two channel members is approximately 180 degrees.

3. The device of claim 2, wherein the latch is capable of engaging the slot in the sidewalls of the second channel member only when the channel members are in an approximately 180 degree position.

4. The device of claim 1, further comprising a post extending inward from a sidewall of the first channel member and a post extending inward from a sidewall of the second channel member.

5. The device of claim 4, wherein the sidewall of the first channel member having a post extending therefrom and the sidewall of the second channel member having a post extending therefrom are adjacent one another.

6. The device of claim 4, further comprising two indents in the locking mechanism aligned so that the posts rest in the indents when the locking mechanism's latch engages the slot in the sidewalls of the second channel member.

7. The device of claim 1, further comprising a tip for dilating an incision or defect in an annulus having distal and proximate ends and four side walls defining a lumen; the proximate end being detachably connected to the second end of the first channel member; the lumen being in communication with the lumen extending through the first and second channel members.

8. The device of claim 7, further comprising an aperture positioned in a sidewall of the tip.

9. The device of claim 7, further comprising one or more flexible arms positioned at the distal end of the tip.

10. The device of claim 9, wherein the flexible arms bend inward towards the lumen of the tip in a relaxed position.

11. The device of claim 9, wherein passage of a prosthetic implant through the tip causes the arms to bend outwards away from the lumen of the tip.

12. The device of claim 9, further comprising a guiding arm positioned at the distal end of the tip, the guiding arm being shorter than the flexible arms.

13. The device of claim 1, further comprising a ratchet mechanism for pushing a prosthetic implant through the first and second channel members, wherein the ratchet mechanism is detachably connected to the second end of the second channel member.

14. The device of claim 13, wherein the ratchet mechanism comprises a handle, a lever pivotally connected to the handle, two sets of clutch plates mounted on the handle, and a plunger engageable by the clutch plates; wherein activating the ratchet mechanism comprises advancing the lever towards the handle.

15. The device of claim 14, wherein the sets of clutch plates are apertured so that the plunger can pass through the clutch plates, and the clutch plates are capable of frictionally engaging the plunger.

16. The device of claim 15, wherein one set of clutch plates are driving clutches that do not frictionally engage the plunger in a resting position; but when the ratchet mechanism is activated by advancing the lever, the driving clutches frictionally engage the plunger and drive the plunger in a distal direction.

17. The device of claim 15, wherein one set of clutch plates are holding clutches that frictionally engage the plunger in a resting position; but when the ratchet mechanism is activated by advancing the lever, the holding clutches do not frictionally engage the plunger.

18. The device of claim 13, wherein detachably connecting the ratchet mechanism to the second end of the second channel member locks the latch of the locking mechanism into the slot in the second channel member.

19. A method of implanting a prosthetic implant in an intervertebral disc space, comprising:
providing a device as in claim 1;
pivoting the first and second channel members to a position substantially less than 180 degrees;
loading the prosthetic implant into the first and second channel members;
pivoting the first and second channel members to a position of approximately 180 degrees;
engaging the latch of the locking mechanism with the slot in the second channel member; and
advancing the prosthetic implant through the first and second channel members and into the intervertebral disc space.

20. The method of claim 19, wherein the prosthetic implant is loaded into the first and second channel members while in a relaxed configuration.

21. The method of claim 20, wherein the prosthetic implant unfolds to an implantation configuration when the first and second channel members are brought to an approximately 180 degree position.

22. The method of claim 21, wherein the prosthetic implant has a substantially smaller cross section in its implantation configuration than in its relaxed configuration.

23. The method of claim 21, wherein the prosthetic implant resumes a relaxed configuration as it exits the device.

24. A device for inserting a prosthetic implant, comprising:
first and second channel members each having first and second ends, a bottom, two sidewalls, and an open side; the second channel member further comprising a slot in the top of its sidewalls positioned along its longitudinal extent; the first and second channel members being pivotally connected at their first ends and defining a lumen extending through the channel members;
a locking mechanism capable of at least partially covering the open sides of the first and second channel members, pivotally connected to the first channel member, and having a latch capable of engaging the slot in the sidewalls of the second channel member;
a tip comprising distal and proximate ends and four side walls defining a lumen, the proximate end being detachably connected to the second end of the first channel member, and the lumen being in communication with the lumen extending through the first and second channel members; and
a ratchet mechanism detachably connected to the second end of the second channel member, comprising a handle, a lever pivotally connected to the handle, two sets of clutch plates mounted on the handle, and a plunger engageable by the clutch plates; wherein activating the ratchet mechanism comprises advancing the lever towards the handle.

25. A method of implanting a prosthetic implant in an intervertebral disc space, comprising:
providing a device as in claim 24;
pivoting the first and second channel members to a position substantially less than 180 degrees;
loading the prosthetic implant into the first and second channel members;
pivoting the first and second channel members to a position of approximately 180 degrees;
engaging the latch of the locking mechanism with the slot in the second channel member;
attaching the tip to the second end of the first channel member and the ratchet mechanism to the second end of the second channel member;
inserting the distal end of the tip into the intervertebral disc space; and
activating the ratchet mechanism by advancing the lever towards the handle to advance the prosthetic implant into the intervertebral disc space.

26. The method of claim 25, wherein the tip is inserted into an incision or defect in the annulus fibrosis of the intervertebral disc space.

27. The method of claim 26, wherein the tip further comprises arms positioned at its distal end that flex outwards to dilate the incision or defect in the annulus as the prosthetic implant passes through the tip.

28. The method of claim 27, wherein the tip further comprises a guiding arm positioned at the distal end of the tip, the guiding arm being shorter than the flexible arms so that the prosthetic implant folds towards the guiding arm as it exits the device.

29. The method of claim 25, wherein the ratchet mechanism is activated to bring the prosthetic implant to a position just before the distal end of the tip prior to inserting the distal end of the tip into the intervertebral disc space.

30. The method of claim 25, wherein the prosthetic implant is loaded into the first and second channel members while in a relaxed configuration.

31. The method of claim 30, wherein the prosthetic implant unfolds to an implantation configuration when the first and second channel members are brought to an approximately 180 degree position.

32. The method of claim 31, wherein the prosthetic implant has a substantially smaller cross sectional area in its implantation configuration than in its relaxed configuration.

33. The method of claim 31, wherein the prosthetic implant resumes a relaxed configuration as it exits the device.
